(19) United States
(12) Patent Application Publication
(10) Pub. No.: US 2007/0154407 A1
(43) Pub. Date: Jul. 5, 2007

(54) INHALER AND STORE FOR A DRY MEDICAMENT FORMULATION AND RELATED METHODS AND USE THEREOF

(75) Inventors: Ralf-Peter PETERS, Bergisch-Gladbach (DE); Gert BLANKENSTEIN, Dortmund (DE)

Correspondence Address: ROBERTS, MLOTKOWSKI & HOBBES, P. O. BOX 10064, MCLEAN, VA, 22102-8064, US

(73) Assignee: BOEHRINGER INGELHEIM INTERNATIONAL GMBH, Ingelheim am Rhein (DE)

(21) Appl. No.: 11/565,235

(22) Filed: Nov. 30, 2006

(30) Foreign Application Priority Data
Dec. 1, 2005 (DE) 10 2005 057 685.0

(51) Int. Cl. A61K 9/14; A61M 11/00

(52) U.S. Cl. 424/46; 128/200.23

(57) ABSTRACT

An inhaler, a store and a method for atomizing a dry medicament formulation are disclosed. The medicament formulation is first dissolved by a liquid solvent and then atomized together with the solvent. This allows easy metering and satisfactory storage of the medicament formulation in the dry state. Particularly preferably, the dry medicament formulation is prepared by drying an initially liquid medicament formulation. Alternatively, the dried medicament formulation may also be expelled in dry form by means of a gas current and atomized.

### BACKGROUND OF THE INVENTION

1. Field of the Invention

The present invention relates to a method of atomizing a dry medicament formulation, an inhaler, a store for a medicament formulation, a method of preparing a dry medicament formulation for an inhaler and a method for producing an aerosol.

2. Description of Related Art

An inhaler is required to atomize an amount of medicament formulation which is defined as accurately as possible, i.e. converted into an aerosol for inhalation. The term “medicament formulation” for the purposes of the present invention includes not only medicaments but therapeutic agents, diagnostic agents or the like and, in particular, all kinds of agents for inhalation.

Two basic types of inhalers are known.

In the so-called dry powder inhaler, the medicament formulation is present in powder form and is expelled and atomized by means of a gas current. Problems often arise in metering the powder, even if it has already been premeasured into individual doses at the factory. During use, i.e., during atomizing, expulsion of all of the respective dose, on the one hand, and atomizing thereof into very fine particles destined for the lungs, on the other hand, may create problems.

Moreover, inhalers for liquid medicament formulations are known, e.g., so-called meter dose inhalers (medicament formulation under gas pressure is expelled through a metering valve and atomized) or so-called soft mist inhalers, as explained for example in the article “Soft Mist Inhalers: A Review of Current Technology”, Michael Hindle, published in “The Drug Delivery Companies Report Autumn/Winter 2004” by PharmaVentures Ltd 2004. The inhalers mentioned therein and the atomizing parameters which can be achieved therewith are thus incorporated herein as a supplementary disclosure and apply accordingly to the present invention insofar as it relates to the atomizing of a liquid and the production of an aerosol from the liquid. With liquid medicament formulations, storage stability is often a problem. Accordingly, undesirable preservatives are then required.

### SUMMARY OF THE INVENTION

An object of the present invention is to provide a method of atomising a dry medicament formulation, an inhaler, a store, a method of preparing a dry medicament formulation and a method for producing an aerosol, in which the advantages of the high storage stability of a dry medicament formulation can be combined with the advantages of simple, defined atomization of a liquid medicament formulation, and/or easier metering of the medicament formulation is made possible.

A first aspect of the present invention comprises first dissolving the dry medicament formulation, particularly for inhalation, with or in a liquid solvent, and then atomizing the solvent with the dissolved medicament formulation, preferably as an aerosol. Thus, on the one hand the advantages of a dry medicament formulation, e.g., good storage stability, and on the other hand, the advantages of a liquid medicament formulation or atomization, namely the achieving of very fine droplet sizes, a low propagation speed and/or other parameters, can be achieved.

One particular advantage is that it is possible to pre-dose the dry medicament formulation at the factory end, for example, where it is not so essential to keep to a precise quantity of solvent for dissolving and delivering a dose of the medicament formulation in the inhaler, i.e. the demands made on the inhaler itself or on the metering accuracy of the solvent are substantially lower. In order to achieve good storage stability, the medicament formulation is preferably only dissolved by the solvent during the atomizing process or immediately beforehand. The solvent may flow continuously or discontinuously through a store for the medicament formulation. This depends particularly on the solution kinetics.

In order to achieve a discontinuous flow of the solvent through the store, or a flow which is slower in relation to the atomizer, for example, in order to allow the medicament formulation to dissolve slowly, suitable valves, capillary stops, timing controls or the like may be used, such as are known from the prior art particularly in conjunction with microfluidic platforms and the handling of fluids.

Particularly preferably, the inhaler comprises a pressure generator, for example, a pump or any other suitable pressure generating means, such as compressed air or liquefied gas, spring force or the like. The store containing the medicament formulation may be arranged upstream or downstream of the pressure generator, as desired. Accordingly, the medicament formulation is dissolved by the solvent which is still under low pressure or already under high pressure and then expelled.

Particularly preferably, the inhaler is portable and/or constructed so that the atomization is effected purely mechanically. This allows ease of handling and provides good operational reliability.

The inhaler or the store may contain the dry medicament formulation, for example, in powder form, pellet form, tablet form or in the form of a lyophilizate. Particularly preferably, however, the medicament formulation is in dried form. The advantages associated with this will be described in more detail hereinafter.

The preparation of the dry medicament formulation is carried out according to a second aspect of the present invention, which may also be realized independently, by filling or metering the initially liquid medicament formulation into the store and then drying it. In particular, the medicament formulation is initially in liquid form or in the form of a solution, the term “solution” here being meant in the broad sense of including, in addition to a preferred solution by chemical definition, a suspension, a mixture of a solution and a suspension or some other preparation, for example.

The initially liquid medicament formulation can very easily be metered, for example using systems which are on the market such as so-called dispensers, pipetting systems, metering pumps or the like which ensure the accurate dosing of tiny quantities of liquid.

Alternatively, or additionally, metering may also be carried out using microfluidic structures, dimensions, effects or the like. For example, a channel or a microstructured region may be filled or covered with the liquid medicament formulation in a quantity determined by the dimensions, particularly automatically by capillary forces, hydrostatic forces or external pressure forces, and in this way accurate metering of the medicament formulation may be obtained.

The medicament formulation may be dried, in particular, by air drying, freeze drying and/or the application of a vacuum.

After drying, a precisely defined quantity or dose of dried medicament formulation is left in the store.

After drying the medicament formulation is preferably hermetically sealed, more particularly sealed so as to be airtight and gastight. For example, the store or individual regions of the store are sealed.

The store can preferably be placed in the inhaler and optionally exchanged. This allows the inhaler to be used a number of times, in particular.

If necessary, the store may comprise a plurality of storage regions for holding the medicament formulation. Each storage region serves to accommodate one dose of the medicament formulation.

Each storage region may have, for example, a microstructured surface, a hydrophilic or hydrophobic section, edge structures or the like, particularly for evening out the surface coverage with the still liquid medicament formulation and/or for measuring or metering the latter.

Particularly preferably, each storage region can be automatically and/or completely filled or covered with the solution by capillary forces.

If required, each storage region may have its own separate outlet or separate nozzle.

It is also possible for the inhaler or store to be designed to deliver two, three or more different medicament formulations—optionally also dry and liquid in any desired combinations—during one atomising process or in successive atomising processes. In particular, the different medicament formulations can then be dissolved and expelled simultaneously or immediately after one another during an atomising process. If different medicament formulations are provided, the store preferably has correspondingly different storage regions.

Particularly preferably, all the storage regions are separate from one another and/or formed by separate storage recesses, irrespective of whether there are several doses of the same medicament formulation and/or different medicament formulations.

Particularly preferably, the storage regions or cavities are formed by recesses, cut outs, depressions or the like in a platform. The still liquid medicament formulation can be fed in, according to one alternative embodiment, before the platform is covered. In this case the platform is only covered after the initially liquid medicament formulation has been dried and is then heat-sealed or otherwise sealed. According to another alternative embodiment platform is closed off or covered before the still liquid medicament formulation is fed in, and this formulation is then supplied through openings, channels or the like which are already present or have to be newly provided. The individual storage regions or cavities can preferably be opened singly, in particular only as required or for dissolving and delivery.

The store is constructed in particular as a cartridge, container, blister, foil, microfluidic platform, strip or disc.

Particularly preferably, the dry or dried medicament is fully dissolved or, if desired, partially dissolved to an intended or defined degree, immediately before its expulsion and atomization by means of a liquid or other solvent, which is passed, in particular, directly through the store or respective storage region. The medicament formulation may be dissolved thereby in the purely chemical sense. However, the result may also be a suspension or other liquefaction or liquid preparation such as a mixture of a suspension and a solution.

The solvent may itself contain or consist of an active substance, activating agent or the like. Alternatively or additionally, different solvents and/or different medicaments may be combined, particularly mixed, during the dissolving process. Accordingly, during or after the dissolving process, at least one activated active substance, active substance mixture, active substance complex or the like may develop, be formed or prepared and delivered.

For example, the dried medicament formulation may contain an active substance which, in the dried state, is immobilized on a carrier particle. Examples of carrier particles include plastic or silicate beads, lactose, hydro gel, polysaccharides such as agarose, reverse micelles or the like. The carrier particles are then resuspended by the solvent but not resolubilised (dissolved). However, it is also possible for the solvent to dissolve or open up these carrier particles. For example, carrier particles such as liposomes can be dissolved by the solvent so that only in this way can the active substance be released and resolubilised (dissolved).

According to a third aspect of the present invention, which can also be realised independently, the dried medicament formulation may also be expelled or atomised in dry form by a gas current. In this case the dried medicament formulation is preferably not immobilised in the relevant storage region but is loosely or movably contained therein, for example. It is also possible for the dried medicament formulation to be in the form of a powder or lyophilizate. The gas current needed for the expulsion or atomization can then be generated, in particular by an air current from a user, e.g., on breathing in or by operating the air pump.

Particularly preferably, the proposed inhaler operates by expelling the respective dose of medicament for a short time—particularly within one to two breaths of the user or within about 1 to 2 seconds. However, it is also possible in principle to use the proposed expulsion for longer-lasting inhalations or for other purposes.

Other aspects, features, properties and advantages of the present invention will become apparent from the following description of preferred embodiments with reference to the drawings.

### BRIEF DESCRIPTION OF THE DRAWINGS

FIG. 1 is a schematic view of a proposed inhaler with a proposed disc-shaped store;

FIG. 2 is a magnified view of a part of the inhaler;

FIG. 3 is a perspective view of the store;

FIG. 4 is a perspective view of a connecting part of the inhaler;

FIG. 5 is a schematic view of a store according to another embodiment;

FIG. 6 is a schematic view of a store according to another embodiment;

FIG. 7 is a schematic view of a store according to yet another embodiment;

FIG. 8 is a schematic view of a store according to an additional embodiment;

FIG. 9 is a block circuit diagram of a proposed method and inhaler according to another embodiment;

FIG. 10 is a block circuit diagram of a proposed method and inhaler according to a further embodiment;

FIG. 11 is a block circuit diagram of a proposed method and inhaler according to an additional embodiment; and

FIG. 12 is a schematic view of an arrangement for simultaneously filling a number of stores.

### DETAILED DESCRIPTION OF THE INVENTION

In the figures, the same reference numerals have been used for the same or similar parts, even if the associated description has been omitted.

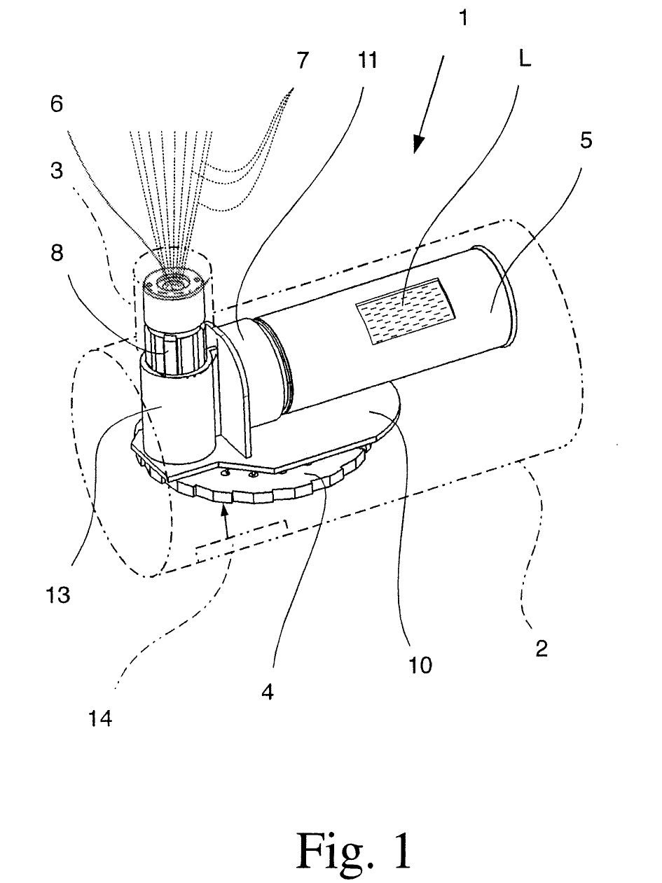

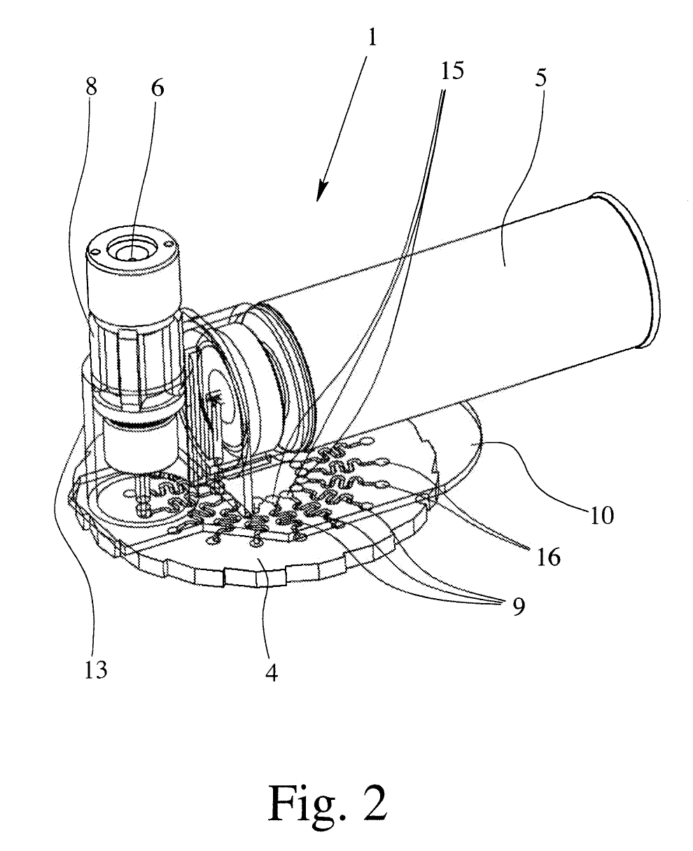

FIG. 1 shows, in a highly schematic view, a proposed inhaler 1 having a housing 2 and mouthpiece 3, which are merely indicated by dotted lines. FIG. 2 shows the inhaler 1 in a magnified view without the housing 2 and mouthpiece 3, but showing a connecting element 10 as transparent, for the purposes of illustration.

The inhaler 1 has a store 4 with a dry medicament formulation, which is not shown in FIGS. 1 & 2. In particular, the store 4 can be inserted in the inhaler 1 and exchanged if necessary.

In the embodiment shown, the store 4 contains a number of doses of the medicament formulation. The inhaler 1 can accordingly be used several times or for several inhalations. The medicament formulation is present in the store 4 in dry form, and more particularly in dried-up form.

The inhaler 1 comprises a reservoir 5, in this example a container or the like, with a liquid solvent L, which is shown in FIG. 1. In particular the inhaler 1 is designed to accommodate the reservoir 5. However, the reservoir 5 may also be integrated in the inhaler 1.

The reservoir 5 may be under a relatively high gas pressure. The solvent L can then be delivered as necessary through a valve (not shown), particularly a metering valve, and specifically is passed first through the store 4 in order to dissolve the medicament formulation to begin with. Because of the gas pressure this solution is then forced through an outlet 6 shown in FIG. 1, such as a nozzle or the like, in an aerosol cloud 7, diagrammatically shown in FIG. 1, into the mouthpiece 3. However, it is also possible for the solvent L or the reservoir 5 not to be under pressure.

In the embodiment shown, the inhaler 1 has a common or appliance-side outlet 6 such as a nozzle or the like. If a number of doses are being delivered, these are all delivered through the same outlet 6. The outlet 6 may optionally also comprise a plurality of outlet channels.

Preferably, the inhaler 1 has a pressure generator 8, for example, a pump or the like, for conveying the solvent L, particularly for delivering it under pressure and atomising it. The pressure generator 8 can suck the solvent L through the store 4, in particular, and then expel it under pressure through the outlet 6. The solvent L together with the dissolved medicament formulation is then atomized into the aerosol cloud 7 in the desired manner.

Preferably, the pressure generator 8 operates purely mechanically. It is also possible, however, for the pressure generator 8 to be operated by gas, spring force, motor-driven, or to operate piezo-electrically, electrically, as a vaporiser or in some other way, for example. The same applies to the inhaler 1 as a whole.

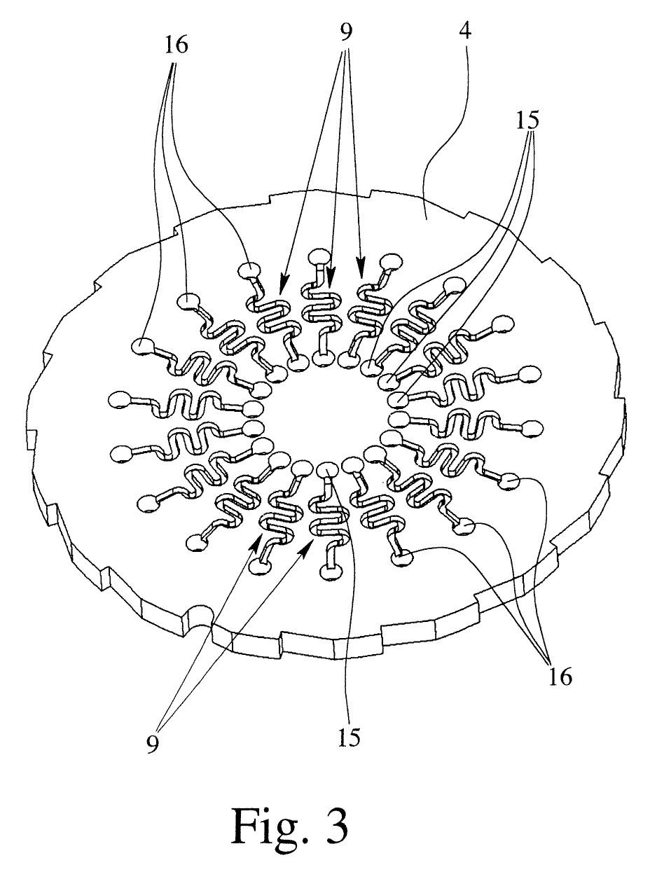

FIG. 3 shows the store 4 which is preferably disc-shaped, annular or wheel-shaped in the embodiment shown, in a perspective schematic view without its associated cover. The store 4 has a number of storage regions 9 each of which serves to receive a dose of the medicament formulation. Each storage region 9 is formed here by a channel or channel section which may preferably extend in a winding or meandering configuration, optionally also in a straight line, spiral or some other pattern.

The store 4 is filled with the desired medicament formulation during manufacture or packaging. The liquid medicament formulation is metered into the individual storage regions 9, in particular. Depending on the design of the storage regions 9 the metering may also be done by simply filling the storage regions 9 which have or hold a specified volume with the (still) liquid medicament formulation. Then the medicament formulation is dried in the storage regions 9.

Finally, the store 4 is closed off by means of a cover (not shown), such as a protective film, seal or the like, made in particular of plastics or metallized plastics and in this way (as far as possible) hermetically sealed, but in particular made fluid-tight and gastight.

In this state, the store 4 can be kept for very long periods. The dried-up medicament formulation has very good storage stability. This can be attributed in particular to the drying process.

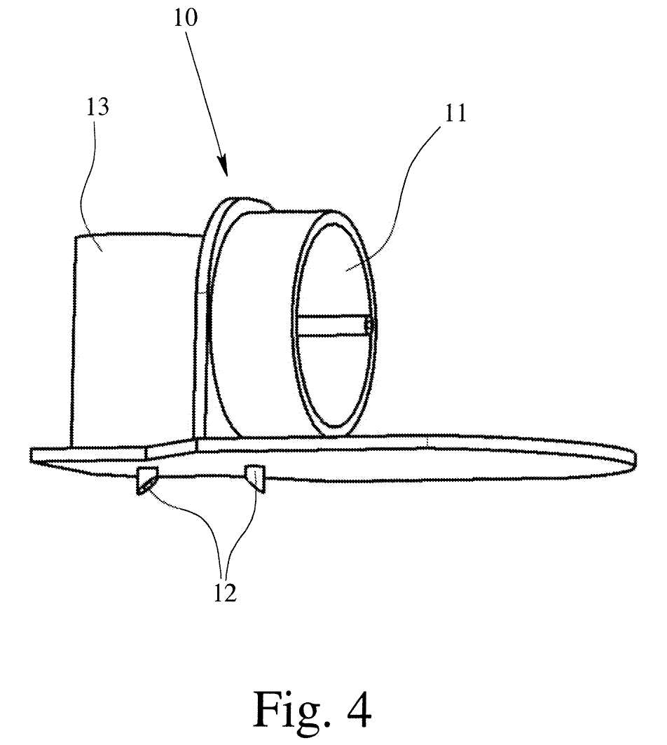

FIG. 4 shows only the connecting element 10 of the inhaler 1 which comprises a receiving region 11 for the reservoir 5 and piercing elements 12 for producing a fluidic connection with the store 4 as required. Moreover, the connecting element 10 is designed for connection or attachment to the outlet 6 or pressure generator 8 and for this purpose has a connecting region 13.

When the inhaler 1 is used, the user (not shown) may for example operate an actuating element 14 for the inhaler 1, shown in FIG. 1. Using a mechanism indicated only by the arrow, the connecting element 10 is then moved or displaced relative to the store 4 such that the piercing elements 12 piece the cover (not shown) of the store 4 and fluidically contact a storage region 9 located in this position. In the embodiment shown this is done by the piercing element 12 which serves to supply the solvent L creating a fluidic connection to the radially inner connecting port 15 of the respective storage region 9 and the other piercing element 12 creating a connection with the corresponding connecting port 16, which is radially outside in this case, in order to discharge the solvent L with the dissolved medicament formulation.

The pressure generation and conveying of the solvent L may be carried out, for example, by first pressing the store 4 against the connecting element 10 so that the nearest storage region 9 is pierced, and then, in the course of a further movement, the connecting region 13 is moved relative to the pressure generator 8 or actuates or initiates the latter in order to achieve the desired conveying and pressure generation. However, other constructional or functional solutions are also possible here.

Moreover, before or after the expulsion of a dose, the store 4, which is rotatable, for example, may be moved on to the next storage region 9, and in particular rotated further. This can be done by actuating the actuating element 14 or in some other way, possibly by manual rotation. The sawtooth edge of the store 4 may cooperate with a locking latch or the like to prevent backward rotation. The sawtooth edge may be used alternatively or in addition to the drive of a counter (not shown). However, it is also possible to drive the counter by some other method.

As a result of the actuation of the actuating element 14, the triggering of the pressure generator 8 or, for example, by the effects of gas pressure or the like, the solvent L then flows out of the reservoir 5 through the storage region 9 to the outlet 6 or pressure generator 8. The dried-up medicament formulation is dissolved by the solvent L and carried along with it.

Tests have shown that comparatively fast dissolving of the medicament formulation is possible depending on various parameters.

Moreover, a relatively long lasting atomization is desired in order to make inhalation easier for the user and thereby increase the probability that the user will inhale a dose as completely as possible. The atomizing process preferably lasts one to two seconds or longer. This ensures that sufficient time is provided for the medicament formulation to dissolve.

However, it is also possible to stop the flow of solvent after the filling of the storage region 9 with the solvent L, in particular for a predetermined time or for the next use, to allow sufficient time for the medicament formulation to dissolve completely or sufficiently.

The medicament formulation dissolved in the solvent L is then atomized together with the solvent L and delivered as an aerosol cloud 7.

In the embodiment shown, the storage cavities or storage regions 9 or channel sections, ports 15, 16 or the like, are preferably formed by depressions in a carrier of the store 4, formed for example by casting, injection molding, deep-drawing or the like, and these cavities or storage regions are covered by the cover (not shown) which has already been mentioned.

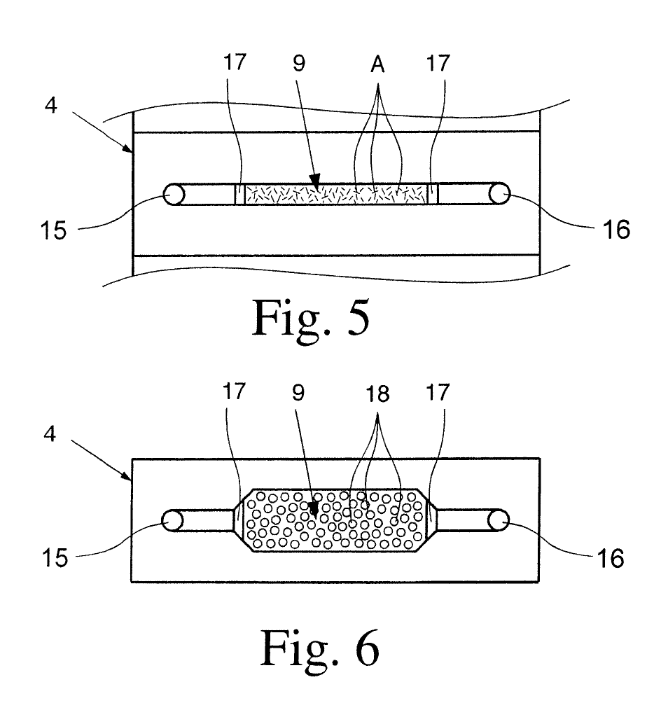

FIG. 5 shows another embodiment of the store 4 without a cover. The medicament formulation A has already been dried in.

The storage region 4 is constructed here as an at least substantially straight channel section. At the beginning and/or end thereof optional capillary stops 17 are formed, e.g., by means of trench-like depressions at right-angles to the longitudinal direction of the channel. These or other stopping structures may, for example, be used to keep the medicament formulation A, which is still in the liquid state, in the storage region 9 before the drying process, i.e., prevent it from flowing out. In an arrangement of this kind the still liquid medicament formulation A can be metered very easily and, for example, directly into the storage region 9 by means of a metering device or the like (not shown). Thus, a defined drying, more particularly in a fixed location, can take place in a storage region 9 which is, in particular, in a favorable location from the flow point of view. In subsequent use, the capillary stops 17 can be easily overcome by the solvent L, particularly as a result of a suitable pressure.

Independently of capillary stops 17, or the like, the storage region 9 can alternatively only be filled with the still liquid medicament formulation A—e.g., through the attachment port 15 or 16—only after the region has been covered with a cover (not shown). The medicament formulation then dries out or is dried out. The attachment ports 15 and 16 are then preferably hermetically sealed after drying.

Preferably, the store 4 consists of a plurality of preferably rigid platforms which each form a storage region 9 with associated attachment ports 15 and 16, which are fixedly or flexibly joined together and form a band, for example. If required, the connections can also be broken, for example, in order to dispose of a platform after the emptying of the respective storage region 9.

In the embodiment shown the store 4 is preferably of belt-, strip- or band-like construction with a plurality of storage regions 9 arranged parallel to one another or one behind the other. In the embodiment shown, the attachment ports 15 and 16 are arranged in the region of the opposing longitudinal edges of the strips. However, other configurations are also possible: for example, the attachment ports 15 and 16 may also be arranged side by side in the region of the same strip edge, when the storage regions are U-shaped.

The storage regions 9 or channel sections may, if necessary, also extend at an angle to the longitudinal direction or substantially parallel to the longitudinal direction or direction of conveying of the store 4.

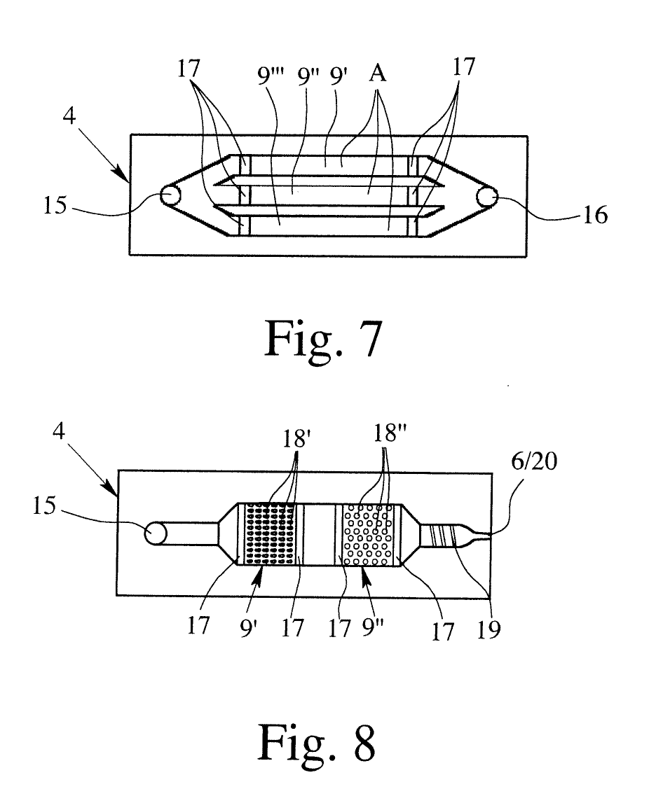

In the other two embodiments that follow, which are described with reference to FIGS. 6 and 7, the forgoing remarks also apply. Accordingly, only the essential differences are mentioned hereinafter.

In the embodiment according to FIG. 6, the storage region 9 is widened. In particular it is a widened channel or channel section. The channel or channel section may, in particular, be constructed as a capillary and/or of such dimensions that capillary forces come into effect. It is even possible to fill the storage region 9 with the solvent L solely by gravitational forces and/or capillary forces. In this case there is no need for pressure generation or for a pressure generator 8 to fill the storage region 9 and dissolve the medicament formulation A, but only for the subsequent delivery and atomization.

Furthermore, elevations, depressions and/or microstructures 18 are preferably formed on a surface (flat side) of the storage region 9, which are, for example, in the form of columns, truncated pyramids or hemispheres. The microstructures 10 may additionally be provided with notches or the like.

The microstructures 18 serve in particular to increase the surface area in order to distribute the dried medicament formulation A over a particularly large surface area and thereby assist or accelerate the drying and later dissolving.

The microstructures 18 may alternatively or additionally serve to cover the surface of the storage region 9 as uniformly as possible with the medicament formulation A. As a result of a uniform coverage, very uniform drying can be achieved with, in particular, at least substantially uniform thickness of the dried medicament formulation A. This contributes to uniform and rapid and/or defined subsequent dissolving of the medicament formulation A.

In the embodiment according to FIG. 7, the store 4 has various storage regions 9′, 9″ and 9′″ for holding different medicament formulations. The different storage regions 9′, 9″ and 9′″ are jointly attached to the associated attachment ports 15 and 16, in parallel in the embodiment shown. However, a serial connection is also possible. Accordingly, in this case, it is even possible for two or three different medicament formulations to be delivered simultaneously in one atomising operation.

Basically, the solvent L and/or the medicament formulation A or the medicament fromulations A may contain an active substance, an activating agent or some other ingredient for forming at least one active substance, medicament or the like after mixing or dissolving with or in the solvent L. This is generally true of all the embodiments.

FIG. 8 shows yet another embodiment of the store 4 with several—in this case two-storage regions 9′ and 9″ arranged one behind the other or connected in series, which contain in particular different medicament formulations A, active substance ingredients, activating agents or the like, preferably again in dry, especially dried-up form.

In particular, only after being dissolved by the solvent L is at least one active substance or medicament formulation prepared or formed and preferably delivered directly as an aerosol 7, as already described.

The channel or channel section—hereinafter referred to as storage cavity for short—which forms the storage region 9 or plurality of storage regions 9, 9′, 9″ or 9′″ preferably has a capacity of from 1 μl to 100 μl, particularly 2 μl to 50 μl, particularly preferably substantially from 2.5 μl to 25 μl.

With the storage cavity being continuously flushed with the solvent for dissolving the medicament formulation or for preparing at least one pharmaceutical or therapeutic active substance or the like, the capacity of the storage cavity is preferably at least substantially 10 to 30% of the total volume of liquid which is delivered in one atomization process.

With the storage cavity or the minimum of one storage region 9 being discontinuously flushed with the solvent L, the volume of the storage cavity is preferably at least or substantially the volume of liquid expelled in each atomization process, i.e. substantially the quantity of solvent L delivered.

The quantity of solvent L delivered or the total delivery amount per atomization process is preferably substantially 5 to 10 μl, particularly 10 to 50 μl.

The individual dose of the medicament formulation is essentially 0.1 to 10 mg, for example, in the case of fenetorol.

The dosage is preferably of a quantity such that total dissolving of the pharmaceutical formulation takes place during a delivery process. In particular, the dosage is at most 50% of the maximum amount that can be dissolved in the respective quantity of solvent to safely ensure total dissolving of the medicament formulation. This preferably also applies to other ingredients, activating agents, proteins or the like.

The storage cavity is preferably oblong, flat and/or narrow in design. This assists, in particular, the diffusion-driven dissolving of the medicament formulation.

Preferably, the depth of the storage cavity is at most 1 mm or significantly less, especially 0.1 to 0.5 mm, the latter range proving advantageous particularly when the flow of solvent through the storage cavity is discontinuous.

The width of the storage cavity is preferably substantially 1 to 5 mm.

The ratio of length to width and/or of length to depth is preferably at least 2, particularly preferably at least 5, most particularly preferably at least 10 or more.

In the embodiment shown, the elevations or microstructures 18′ of the first storage region 9′ are preferably teardrop-shaped, at least in the plan view shown. The elevations or microstructures 18″ of the second storage region, by contrast, are preferably column-shaped, particularly at least substantially cylindrical, if necessary with rounded ends, in the embodiment shown.

If necessary, the elevations or microstructures 18′ and 18″ are conical or tapering towards the free end and/or are provided with lateral recesses, notches, longitudinal grooves or the like.

The elevations or microstructures 18′ have a length of 0.2 to 0.3 mm at their base and from about 0.15 to 0.2 mm at their free end, in the embodiment according to FIG. 8. The width is, in particular, substantially 10 to 100 μm.

The central spacing is preferably substantially 0.2 to 0.3 mm in the longitudinal direction and/or 0.1 to 0.2 mm in the transverse direction in the embodiment shown.

In the case of a columnar construction, the diameter of the elevations or microstructures 18″ is preferably substantially 10 to 200 μm, the central spacing in the longitudinal direction is substantially 0.1 to 0.2 mm and in the transverse direction 0.1 to 0.2 mm.

The height of the elevations or microstructures 18 may correspond to 10 to 100%, more particularly, substantially 20 to 60% of the depth of the storage cavity.

The elevations or microstructures 18 may significantly increase the ratio of surface area to volume, in particular, by at least a factor 5 and, more preferably, by substantially a factor of 8 to 15.

Preferably, the inhaler 1 or store 4 has a mixing device 19 as shown, by way of example, in FIG. 8. The mixing device 19 serves in particular for mixing the solvent with the medicament A transported thereby, particularly preferably in order to achieve a homogeneous or more homogeneous mixture, solution, suspension or the like.

The mixing device 19 is provided downstream of the storage region 9. In the present embodiment, mixing device 19 is provided downstream of the two storage regions 9′ and 9″. It may additionally or alternatively be arranged between the two storage regions 9′ and 9″.

In the embodiment shown, the mixing device 19 is integrated in particular in the store 4. Preferably, a separate mixing device 19 is associated with each store 4 or storage region 9.

However, it is also possible, in principle, to provide a common mixing device 19 through which a plurality of doses can be delivered one after the other. In this case, the mixing device 19 is preferably associated with or mounted upstream of or integrated in a common outlet 6 of the inhaler 1.

The mixing device 19 is, in particular, a static mixer. Preferably, it is a micromixer, more particularly as published in the article “Micromixers—a review” by Nam-Trung Nguven et al., J. Micromech. Microeng. 15 (2005) R1-R16.

Basically, the mixing device 19 may be a device for producing turbulence. In the embodiment shown the mixing device 19 preferably comprises a plurality of sloping elevations, strips or the like.

The store 4 or each individual platform of the store 4 may, if necessary, comprise an outlet 6, particularly a nozzle 20, for direct delivery, particularly atomization of the solvent L with the dissolved medicament formulation and expulsion thereof in the form of an aerosol (not shown). In particular, the individual doses are then delivered through separate outlets 6 or nozzles 20.

The described arrangement of the reservoir 5 for the solvent L on the inhaler 1 is merely a particularly preferred alternative embodiment. Alternatively or additionally, it is possible to provide the store 4 with at least one reservoir 5 for the solvent L. According to a first variant, a central or common reservoir 5, for example, may be provided for the solvent L for a number of doses of the medicament formulation A. The solvent L is then taken as required. According to a second alternative embodiment, a plurality of reservoirs 5 may be provided, each containing a predefined quantity or dose of solvent L and being associated, in particular, with a specific storage region 9 containing a dose of the medicament formulation A. In this case, in order to dissolve a dose of the medicament formulation A, the solvent L is taken from an associated reservoir 5 in each case so that, thanks to the pre-dosing of the solvent L, there is no further need to meter the solvent L through the inhaler 1.

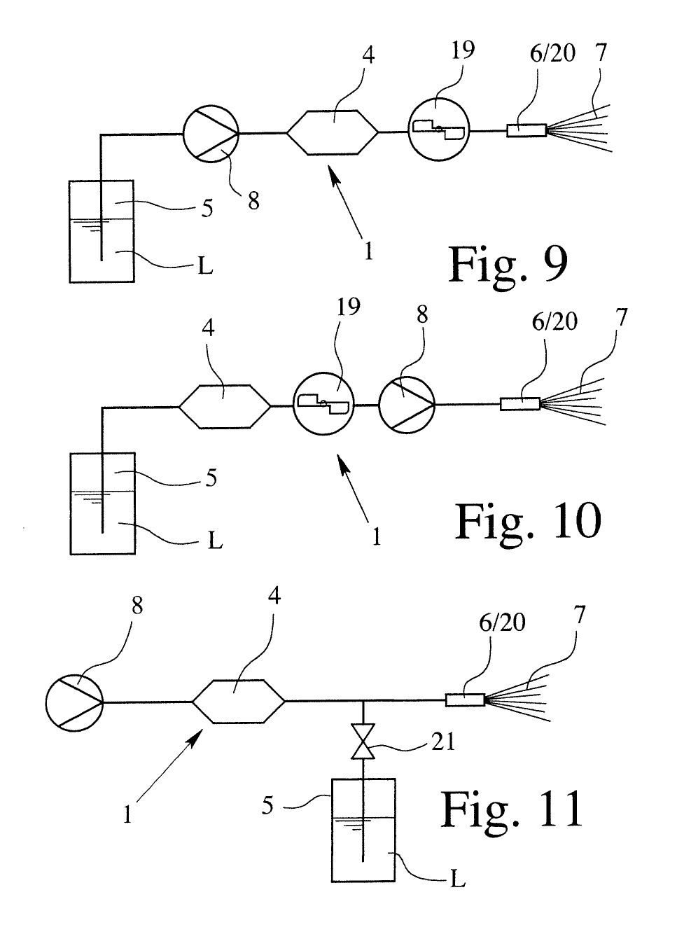

Basically, various arrangements are possible for carrying out the proposed method and constructing the proposed inhaler 1, as will be described in more detail hereinafter with reference to the block circuit diagrams of various embodiments according to FIGS. 9 to 11.

In the embodiment according to FIG. 9, the pressure generator 8 is arranged between the reservoir 5 and the store 4, i.e., the store 4 is mounted downstream of the pressure generator 8. The store 4 or its storage region 9 (not shown) containing the initially dry medicament formulation is thus arranged at the high pressure end and for the dissolving process and subsequent delivery it is acted upon by solvent L under high pressure or said solvent L flows through it.

The optional mixing device 19 is provided downstream of the store 4, if provided.

Finally, the medicament formulation dissolved by the solvent L is atomized through the outlet 6 or nozzle 20 in the form of an aerosol 7, which is expelled.

Instead of the pressure generator 8, any other pressure generating process may be carried out. In particular, the solvent L may also be under or be placed under gas pressure and/or spring pressure. In this case, also, the store 4 is acted upon by solvent L under high pressure or delivery pressure or said solvent L flows through it.

In the embodiment according to FIG. 10 the store 4 and the optional mixing device 19 are arranged upstream of the pressure generator 8 and are accordingly acted upon or flushed through only by solvent L under low pressure. In this case, however, the optional mixing device 19 may also be arranged downstream of the pressure generator 8 or integrated therein.

The embodiments according to FIGS. 9 and 10 are particularly suitable for a continuous flow—i.e., a continuous throughflow—of the solvent L through the store 4 or respective storage region 9 and for immediately subsequent delivery or atomization. However, it is also theoretically possible for the flow to be discontinuous or at least initially slowed down.

In the embodiment according to FIG. 11, the reservoir 5 containing the solvent L is fluidically connected between the store 4 and the outlet 6 or nozzle 20. By means of the pressure generator 8 or some other pump or the like (not shown) the solvent L can be sucked or conveyed out of the reservoir 5 initially into the store 4. Then the direction of flow is reversed. The pressure generator 8 then places the solvent L containing the dissolved medicament formulation under pressure, so that the desired delivery takes place through the outlet 6 or nozzle 20 in the form of an aerosol 7. Meanwhile, unwanted backflow of the solvent L, which is preferably under high pressure, into the reservoir 5 is prevented by means of a suitable valve 21, particularly a non-return valve or one-way valve.

In the embodiment according to FIG. 11, in particular, the pressure and/or the flow velocity of the solvent L for filling the store 4 and dissolving the medicament formulation may be reduced, possibly significantly, compared with the relatively high pressure for the expulsion and atomization process.

In particular, the embodiment according to FIG. 11 makes it possible not only to slow down but also to stop the flow of solvent in the store 4 in order to be able to ensure total dissolving of the medicament formation in the store 4. Particularly preferably, the uptake of the solvent L takes place independently of the expulsion or with other parameters, particularly with a different speed, different flow volume, different pressure and/or the like.

Particularly in the case of substances or medicament formulations which are not easily soluble, the store 4 is filled with the solvent L particularly slowly or the solvent L flows through it particularly slowly and/or the current of solvent is stopped, i.e., the delay time within the respective storage region 9 is increased so as to ensure that the medicament is dissolved as much as possible or substantially.

Additionally or alternatively, it is advantageous to carry out the mixing by means of the mixing device 19, by the use of turbulence or the like in the case of substances or medicament formulations which are not easily soluble, and/or in order to produce uniform suspensions or the like.

The flow volume of the solvent L is preferably 5 to 50 μl per second, in particular at least during the atomization and optionally also during the continuous flow through the store 4, i.e., during the dissolving of the medicament formulation.

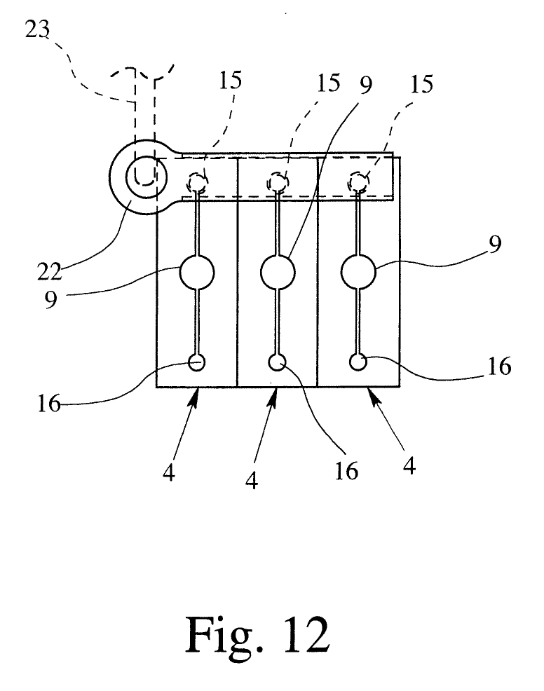

As already mentioned, the medicament formulation in the store 4 is preferably present in dried-up form, particularly as a coating on a surface. However, the medicament formulation may theoretically also be present in the form of a powder, tablet, bead, gel or the like. A preferred method of introducing the initially liquid medicament formulation for simultaneously filling a number of storage regions 9 will now be described with reference to FIG. 12.

Several storage regions 9 of a store 4 or of several stores 4 are connected, particularly in parallel or possible in series, to a filling device 22. In the embodiment shown the filling device 22 is fluidically in contact with the respective storage regions 9 through the connecting ports 15. However, the filling device 22 may, for example, also be used directly for filling the storage regions 9, especially when the storage regions 9 have not yet been covered.

The filling device 22 is in turn connected or connectable to a feed device 23, such as a pipetting device, metering pump or other means for feeding the (still) liquid medicament formulation (not shown) into the storage regions 9.

Particularly preferably, the medicament formulation is supplied in unmetered form, i.e., not in doses tailored to the individual storage regions 9. Rather, the individual storage regions 9 or the respective store 4 is or are constructed so that only a defined, desired quantity of the medicament formulation remains in the respective storage region 9, for example, as a result of corresponding capillary forces or dimensions. In particular, this can be achieved by means of the combinations of capillary stops 17 and microstructures 18 and/or other structures and/or dimensions of the storage regions 9, as indicated in FIGS. 6 to 8.

Then, the initially still liquid medicament formulation is dried in the individual storage regions 9. This can be carried out at elevated temperature, reduced pressure and/or by freeze drying to speed up the process.

In the embodiment shown in FIG. 12, the filling device 22 is put on only temporarily and removed again after filling. However, the filling device 22 may theoretically also be integrated in the storage device 4 and/or fixedly attached thereto, possibly even formed by a common channel or the like.

Individual features and aspects of the various embodiments may also be combined with one another as desired. Accordingly, the embodiments may also be combined with one another as desired.

Some preferred ingredients and/or formulations of the medicament formulation A are listed below. In particular, the still liquid or already dissolved medicament formulation A may consist of aqueous or non-aqueous solutions, mixtures, suspensions, ethanol-containing or solvent-free formulations or the like.

The below mentioned compounds may be used on their own or combined with other active substances for use in the device according to this invention. These include, in particular, betamimetics, anticholinergics, corticosteroids, PDE4-inhibitors, LTD4-antagonists, EGFR-inhibitors, dopamin-agonists, antiallergic agents, PAF-antagonists und P13-kinase inhibitors, but also combinations of two or three active substances, i.e,:

Betamimetics with corticosteroids, PDE4-inhibitors, EGFR-inhibitors or LTD4-antagonists,

Anticholinergics with betamimetics, corticosteroids, PDE4-inhibitors, EGFR-inhibitors or LTD4-antagonists,

Corticosteroids with PDE4-inhibitors, EGFR-inhibitors or LTD4-antagonists

PDE4-inhibitors with EGFR-inhibitors or LTD4-antagonists

EGFR-inhibitors with LTD4-antagonists.

Examples of preferred betamimetics which may be mentioned include Albuterole, Arformoterole, Bambuterole, Bitolterole, Broxaterole, Carbuterole, Clenbuterole, Fenoterole, Formoterole, Hexoprenaline, Ibuterole, Isoetharine, Isoprenaline, Levosalbutamole, Mabuterole, Meluadrine, Metaproterenole, Orciprenaline, Pirbuterole, Procaterole, Reproterole, Rimiterole, Ritodrine, Salmefamole, Salmeterole, Soterenole, Sulphonterole, Terbutaline, Tiaramide, Tolubuterole, Zinterole, CHF-1035, HOKU-81, KUL-1248 and
3-(4-{6-[2-Hydroxy-2-(4-hydroxy-3-hydroxymethyl-phenyl)-ethylamino]-hexyloxy}-butyl)-benzyl-sulfonamide
5-[2-(5,6-Diethyl-indan-2-ylamino)-1-hydroxy-ethyl]-8-hydroxy-1H-quinolin-2-one
4-Hydroxy-7-[2-{[2-{[3-(2-phenylethoxy)propyl]sulphonyl}ethyl]-amino}ethyl]-2(3H)-benzothiazolone
1-(2-Fluoro-4-hydroxyphenyl)-2-[4-(1-benzimidazolyl)-2-methyl-2-butylamino]ethanole
1-[3-(4-Methoxybenzyl-amino)-4-hydroxyphenyl]-2-[4-(1-benzimidazolyl)-2-methyl-2-butylamino]ethanole
1-[2H-5-hydroxy-3-oxo-4H-1,4-benzoxazin-8-yl]-2-[3-(4-N,N-dimethylaminophenyl)-2-methyl-2-propylamino]ethanole
1-[2H-5-hydroxy-3-oxo-4H-1,4-benzoxazin-8-yl]-2-[3-(4-methoxyphenyl)-2-methyl-2-propylamino]ethanole
1-[2H-5-hydroxy-3-oxo-4H-1,4-benzoxazin-8-yl]-2-[3-(4-n-butyloxyphenyl)-2-methyl-2-propylamino]ethanole
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-Hydroxy-8-(1-hydroxy-2-isopropylaminobutyl)-2H-1,4-benzoxazin-3-(4H)-one
1-(4-Amino-3-chloro-5-trifluormethylphenyl)-2-tert.-butylamino)ethanol
6-Hydroxy-8-{1-hydroxy-2-[2-(4-methoxy-phenyl)-1,1-dimethyl-ethylamino]-ethyl}-4H-benzo[1,4]oxazin-3-one
6-Hydroxy-8-{1-hydroxy-2-[2-(4-phenoxy-acetic acid ethylester)-1,1-dimethyl-ethylamino]-ethyl}-4H-benzo[1,4]oxazin-3-one
6-Hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-Dimethyl-2-(2,4,6-trimethylphenyl)-ethylamino]-1-hydroxy-ethyl}-6-hydroxy-4H-benzo[1,4]oxazin-3-one
6-Hydroxy-8-{1-hydroxy-2-[2-(4-hydroxy-phenyl)-1,1-dimethyl-ethylamino]-ethyl 3-4H-benzo[1,4]oxazin-3-one
6-Hydroxy-8-{1-hydroxy-2-[2-(4-isopropyl-phenyl)-1,1dimethyl-ethylamino]-ethyl}-4H-benzo[1,4]oxazin-3-one
8-{2-[2-(4-Ethyl-phenyl)-1,1-dimethyl-ethylamino]-1-hydroxy-ethyl}-6-hydroxy-4H-benzo[1,4]oxazin-3-one
8-{2-[2-(4-Ethoxy-phenyl)-1,1-dimethyl-ethylamino]-1-hydroxy-ethyl}-6-hydroxy-4H-benzo[1,4]oxazin-3-one
4-(4-{2-[2-Hydroxy-2-(6-hydroxy-3-oxo-3,4-dihydro-2H-benzo[1,4]oxazin-8-yl)-ethylamino]2-methyl-propyl}-phenoxy)-butyric acid
8-{2-[2-(3,4-Difluor-phenyl)-1,1-dimethyl-ethylamino]-1-hydroxy-ethyl}-6-hydroxy-4H-benzo[1,4]oxazin-3-on
1-(4-Ethoxy-carbonylamino-3-cyano-5-fluorophenyl)-2-(tert.-butylamino)ethanol
2-Hydroxy-5-(1-hydroxy-2-{2-[4-(2-hydroxy-2-phenyl-ethylamino)-phenyl]-ethylamino}-ethyl)-benzaldehyde
N-[2-Hydroxy-5-(1-hydroxy-2-{2-[4-(2-hydroxy-2-phenyl-ethylamino)-phenyl]-ethylamino}-ethyl)-phenyl]-formamide
8-Hydroxy-5-(1-hydroxy-2-{2-[4-(6-methoxy-biphenyl-3-ylamino)-phenyl]-ethylamino}-ethyl)-1H-quinolin-2-one
8-Hydroxy-5-[1-hydroxy-2-(6-phenethylamino-hexylamino)-ethyl]-1H-quinolin-2-one
5-[2-(2-{4-[4-(2-Amino-2-methyl-propoxy)-phenylamino]-phenyl}-ethylamino)-1-hydroxy-ethyl]-8-hydroxy-1H-quinolin-2-one
[3-(4-{6-[2-Hydroxy-2-(4-hydroxy-3-hydroxymethyl-phenyl)-ethylamino]-hexyloxy}-butyl)-5-methyl-phenyl]-urea
4-(2-{6-[2-(2,6-Dichloro-benzyloxy)-ethoxy]-hexylamino}-1-hydroxy-ethyl)-2-hydroxymethyl-phenol
3-(4-{6-[2-Hydroxy-2-(4-hydroxy-3-hydroxymethyl-phenyl)-ethylamino]-hexyloxy}-butyl)-benzenesulfonamide
3-(3-{7-[2-Hydroxy-2-(4-hydroxy-3-hydroxymethyl-phenyl)-ethylamino]-heptyloxy}-propyl)-benzenesulfonamide
4-(2-{6-[4-(3-Cyclopentanesulfonyl-phenyl)-butoxy]-hexylamino}-1-hydroxy-ethyl)-2-hydroxymethyl-phenol
N-Adamantan-2-yl-2-(3-{2-[2-hydroxy-2-(4-hydroxy-3-hydroxymethyl-phenyl)-ethylamino]-propyl}-phenyl)-acetamide
   optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate.

Examples of preferred anticholinergics which may be mentioned include Tiotropium salts, preferred the bromide salt, Oxitropium salts, preferred the bromide salt, Flutropium salts, preferred the bromide salt, Ipratropium salts, preferred the bromide salt, Glycopyrronium salts, preferred the bromide salt, Trospium salts, preferred the chloride salt, Tolterodin. From the above mentioned salts the pharmacologically active part is the cation, possible anions are chloride, bromide, iodide, sulfate, phosphate, methansulfonate, nitrate, maleate, acetate, citrate, fumarate, tartrate, oxalate, succinate, benzoate oder p-toluenesulfonate. Furthermore
2,2-Diphenylpropion acid tropenolester-methobromide
2,2-Diphenylpropion acid scopinester-methobromide
2-Fluor-2,2-Diphenylacetic acid scopinester-methobromide
2-Fluor-2,2-Diphenylacetic acid tropenolester-methobromide
3,3′,4,4′-Tetrafluorbenzil acid tropenolester-Methobromide
3,3′,4,4′-Tetrafluorbenzil acid scopinester-Methobromide
4,4′-Difluorbenzil acid tropenolester-Methobromide
4,4′-Difluorbenzil acid scopinester-Methobromide
3,3′-Difluorbenzil acid tropenolester-Methobromide
3,3′-Difluorbenzil acid scopinester-Methobromide
9-Hydroxy-fluoren-9-carbon acid tropenolester-Methobromide
9-Fluor-fluoren-9-carbon acid tropenolester-Methobromide
9-Hydroxy-fluoren-9-carbon acid scopinester-Methobromide
9-Fluor-fluoren-9-carbon acid scopinester Methobromide
9-Methyl-fluoren-9-carbon acid tropenolesterMethobromide
9-Methyl-fluoren-9-carbon acid scopinesterMethobromide
Benzil acid cyclopropyltropinester-Methobromide
2,2-Diphenylpropion acid cyclopropyltropinester-Methobromide
9-Hydroxy-xanthen-9-carbon acid cyclopropyltropinesterMethobromide
9-Methyl-fluoren-9-carbon acid cyclopropyltropinester-Methobromide
9-Methyl-xanthen-9-carbon acid cyclopropyltropinester-Methobromide
9-Hydroxy-fluoren-9-carbon acid cyclopropyltropinester-Methobromide
4,4′-Difluorbenzil acid methylestercyclopropyltropinester-Methobromide
9-Hydroxy-xanthen-9-carbon acid tropenolester-Methobromide
9-Hydroxy-xanthen-9-carbon acid scopinester Methobromide
9-Methyl-xanthen-9-carbon acid tropenolester-Methobromide
9-Methyl-xanthen-9-carbon acid scopinesterMethobromide
9-Ethyl-xanthen-9-carbon acid tropenolester Methobromide
9-Difluormethyl-xanthen-9-carbon acid tropenolester-Methobromide
9-Hydroxymethyl-xanthen-9-carbon acid scopinester-Methobromide

Examples of preferred corticosteroids which may be mentioned include Beclomethasone, Betamethasone, Budesonide, Butixocorte, Ciclesonide, Deflazacorte, Dexamethasone, Etiprednole, Flunisolide, Fluticasone, Loteprednole, Mometasone, Prednisolone, Prednisone, Rofleponide, Triamcinolone, RPR-106541, NS-126, ST-26 and
6,9-Difluoro-17-[(2-furanylcarbonyl)oxy]-11-hydroxy-16-methyl-3-oxo-androsta-1,4-dien-17-carbothion acid (S)-fluoromethylester
6,9-Difluoro-11-hydroxy-16-methyl-3-oxo-17-propionyloxy-androsta-1,4-dien-17-carbothion acid (S)—(2-oxo-tetrahydro-furan-3S-yl)ester,
6α,9α-difluoro-11β-hydroxy-16α-methyl-3-oxo-17α-(2,2,3,3-tertamethylcyclo-propylcarbonyl)oxy-androsta-1,4-diene-17β-carboxylic acid cyanomethyl ester optionally in racemic form, as enantiomers, diastereomeres or as pharmacologically acceptable salts, solvates or hydrates. Examples for preferred salts and derivatives are alkali salts, i.e. sodium or potassium salts, sulfobenzoates, phosphates, isonicotinates, acetates, dichloroacetates, propionates, dihydrogenphosphates, palmitates, pivalates or furoates.

Examples of preferred PDE4-inhibtors which may be mentioned include Enprofylline, Theophylline, Roflumilaste, Ariflo (Cilomilast), Tofimilaste, Pumafentrine, Lirimilaste, Arofylline, Atizorame, D-4418, Bay-198004, BY343, CP-325,366, D-4396 (Sch-351591), AWD-12-281 (GW-842470), NCS-613, CDP-840, D-4418, PD-168787, T-440, T-2585, V-11294A, Cl-1018, CDC-801, CDC-3052, D-22888, YM-58997, Z-15370 and
N-(3,5-Dichloro-1-oxo-pyridin-4-yl)-4-difluoromethoxy-3-cyclopropylmethoxy-benzamide
(−)p-[(4aR*, 10bS*)-9-Ethoxy-1,2,3,4,4a,10b-hexahydro-8-methoxy-2-methylbenzo[s][1,6]naphthyridin-6-yl]-N,N-diisopropylbenzamid
(R)—(+)-1-(4-Bromobenzyl)-4-[(3-cyclopentyloxy)-4-methoxyphenyl]-2-pyrrolidon
3-(Cyclopentyloxy-4-methoxyphenyl)-1-(4-N′-[N-2-cyano-5-methyl-isothioureido]benzyl)-2-pyrrolidone
cis[4-Cyano-4-(3-cyclopentyloxy-4-methoxyphenyl)cyclohexan-1-carbon acid]
2-carbomethoxy-4-cyano-4-(3-cyclopropylmethoxy-4-difluoromethoxy-phenyl)cyclohexan-1-one
cis[4-Cyano-4-(3-cyclopropylmethoxy-4-difluoromethoxyphenyl)cyclohexan-1-ol]
(R)—(+)-Ethyl[4-(3-cyclopentyloxy-4-methoxyphenyl)pyrrolidin-2-yliden]acetate
(S)—(−)-Ethyl[4-(3-cyclopentyloxy-4-methoxyphenyl)pyrrolidin-2-yliden]acetate
9-Cyclopentyl-5,6-dihydro-7-ethyl-3-(2-thienyl)-9H-pyrazolo[3,4-c]-1,2,4-triazolo[4,3-a]pyridine
9-Cyclopentyl-5,6-dihydro-7-ethyl-3-(tert-butyl)-9H-pyrazolo[3,4-c]-1,2,4-triazolo[4,3-a]pyridine
   optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate.

Examples of preferred LTD4-antagonists which may be mentioned include Montelukaste, Pranlukaste, Zafirlukaste, MCC-847 (ZD-3523), MN-001, MEN-91507 (LM-1507), VUF-5078, VUF-K-8707, L-733321 and
1-(((R)—(3-(2-(6,7-Difluoro-2-quinolinyl)ethenyl)phenyl)-3-(2-(2-hydroxy-2-propyl)phenyl)thio)methylcyclopropane-acetic acid,
1-(((1 (R)-3(3-(2-(2,3-Dichlorothieno[3,2-b]pyridin-5-yl)-(E)-ethenyl)phenyl)-3-(2-(1-hydroxy-1-methylethyl)phenyl)propyl)thio)methyl)cyclopropane acetic acid
[2-[[2-(4-tert-Butyl-2-thiazolyl)-5-benzofuranyl]oxymethyl]phenyl}acetic acid
   optionally, in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate. Further examples for optionally preferred salts and derivatives are alkali salts, i.e. sodium or potassium salts, sulfobenzoates, phosphates, isonicotinates, acetates, propionates, dihydrogenphosphates, palmitates, pivalates or furoates.

Examples of preferred EGFR-inhibtors which may be mentioned include Cetuximabe, Trastuzumabe, ABX-EGF, Mab ICR-62 and
4-[(3-Chlor-4-fluorphenyl)amino]-6-{[4-(morpholin-4-yl)-1-oxo-2-buten-1-yl]-amino}-7-cyclopropylmethoxy-chinazoline
4-[(3-Chlor-4-fluorphenyl)amino]-6-{[4-(N,N-diethylamino)-1-oxo-2-buten-1-yl]-amino}-7-cyclopropylmethoxy-chinazoline
4-[(3-Chlor-4-fluorphenyl)amino]-6-{[4-(N,N-dimethylamino)-1-oxo-2-buten-1-yl]amino}-7-cyclopropylmethoxy-chinazoline
4-[(R)—(1-Phenyl-ethyl)amino]-6-{[4-(morpholin-4-yl)-1-oxo-2-buten-1-yl]amino}-7-cyclopentyloxy-chinazoline
4-[(3-Chlor-4-fluor-phenyl)amino]-6-{[4-((R)-6-methyl-2-oxo-morpholin-4-yl)-1-oxo-2-buten-1-yl]amino}-7-cyclopropylmethoxy-chinazoline
4-[(3-Chlor-4-fluor-phenyl)amino]-6-{[4-((R)-6-methyl-2-oxo-morpholin-4-yl)-1-oxo-2-buten-1-yl]amino}-7-[(S)—(tetrahydrofuran-3-yl)oxy]-chinazoline
4-[(3-Chlor-4-fluor-phenyl)amino]-6-{[4-((R)-2-methoxymethyl-6-oxo-morpholin-4-yl)-1-oxo-2-buten-1-yl]amino}-7-cyclopropylmethoxy-chinazoline
4-[(3-Chlor-4-fluor-phenyl)amino]-6-[2-((S)-6-methyl-2-oxo-morpholin-4-yl)-ethoxy]-7-methoxy-chinazoline
4-[(3-Chlor-4-fluorphenyl)amino]-6-({4-[N-(2-methoxy-ethyl)-N-methyl-amino]-1-oxo-2-buten-1-yl}amino)-7-cyclopropylmethoxy-chinazoline
4-[(3-Chlor-4-fluorphenyl)amino]-6-{[4-(N,N-dimethylamino)-1-oxo-2-buten-1-yl]amino}-7-cyclopentyloxy-chinazoline
4-[(R)—(1-Phenyl-ethyl)amino]-6-{[4-(N,N-bis-(2-methoxy-ethyl)-amino)-1-oxo-2-buten-1-yl]amino}-7-cyclopropylmethoxy-chinazoline
4-[(R)—(1-Phenyl-ethyl)amino]-6-({4-[N-(2-methoxy-ethyl)-N-ethyl-amino]-1-oxo-2-buten-1-yl}amino)-7-cyclopropylmethoxy-chinazoline
4-[(R)—(1-Phenyl-ethyl)amino]-6-({4-[N-(2-methoxy-ethyl)-N-methyl-amino]-1-oxo-2-buten-1-yl}amino)-7-cyclopropylmethoxy-chinazoline
4-[(R)—(1-Phenyl-ethyl)amino]-6-({4-[N-(tetrahydropyran-4-yl)-N-methyl-amino]-1-oxo-2-buten-1-yl}amino)-7-cyclopropylmethoxy-chinazoline
4-[(3-Chlor-4-fluorphenyl)amino]-6-{[4-(N,N-dimethylamino)-1-oxo-2-buten-1-yl]amino}-7-((R)-tetrahydrofuran-3-yloxy)-chinazoline
4-[(3-Chlor-4-fluorphenyl)amino]-6-{[4-(N,N-dimethylamino)-1-oxo-2-buten-1-yl]amino}-7-((S)-tetrahydrofuran-3-yloxy)-chinazoline
4-[(3-Chlor-4-fluorphenyl)amino]-6-({4-[N-(2-methoxy-ethyl)-N-methyl-amino]-1-oxo-2-buten-1-yl}amino)-7-cyclopentyloxy-chinazoline
4-[(3-Chlor-4-fluorphenyl)amino]-6-{[4-(N-cyclopropyl-N-methyl-amino)-1-oxo-2-buten-1-yl]amino}-7-cyclopentyloxy-chinazoline
4-[(3-Chlor-4-fluorphenyl)amino]-6-{[4-(N,N-dimethylamino)-1-oxo-2-buten-1-yl]amino}-7-[(R)—(tetrahydrofuran-2-yl)methoxy]-chinazoline
4-[(3-Chlor-4-fluorphenyl)amino]-6-{[4-(N,N-dimethylamino)-1-oxo-2-buten-1-yl]amino}-7-[(S)—(tetrahydrofuran-2-yl)methoxy]-chinazoline
4-[(3-Ethinyl-phenyl)amino]-6,7-bis-(2-methoxy-ethoxy)-chinazoline
4-[(3-Chlor-4-fluorphenyl)amino]-7-[3-(morpholin-4-yl)-propyloxy]-6-[(vinyl-carbonyl)amino]-chinazoline
4-[(R)—(1-Phenyl-ethyl)amino]-6-(4-hydroxy-phenyl)-7H-pyrrolo[2,3-d]pyrimidine
3-Cyano-4-[(3-chlor-4-fluorphenyl)amino]-6-{[4-(N,N-dimethylamino)-1-oxo-2-buten-1-yl]amino}-7-ethoxy-chinoline
4-{[3-Chlor-4-(3-fluor-benzyloxy)-phenyl]amino}-6-(5-{[(2-methansulfonyl-ethyl)amino]methyl}-furan-2-yl)chinazoline
4-[(R)—(1-Phenyl-ethyl)amino]-6-{[4-((R)-6-methyl-2-oxo-morpholin-4-yl)-1-oxo-2-buten-1-yl]amino}-7-methoxy-chinazoline
4-[(3-Chlor-4-fluorphenyl)amino]-6-{[4-(morpholin-4-yl)-1-oxo-2-buten-1-yl]-amino}-7-[(tetrahydrofuran-2-yl)methoxy]-chinazoline
4-[(3-Chlor-4-fluorphenyl)amino]-6-({4-[N,N-bis-(2-methoxy-ethyl)-amino]-1-oxo-2-buten-1-yl}amino)-7-[(tetrahydrofuran-2-yl)methoxy]-chinazoline
4-[(3-Ethinyl-phenyl)amino]-6-{[4-(5,5-dimethyl-2-oxo-morpholin-4-yl)-1-oxo-2-buten-1-yl]amino}-chinazoline
4-[(3-Chlor-4-fluor-phenyl)amino]-6-[2-(2,2-dimethyl-6-oxo-morpholin-4-yl)-ethoxy]-7-methoxy-chinazoline
4-[(3-Chlor-4-fluor-phenyl)amino]-6-[2-(2,2-dimethyl-6-oxo-morpholin-4-yl)-ethoxy]-7-[(R)—(tetrahydrofuran-2-yl)methoxy]-chinazoline
4-[(3-Chlor-4-fluor-phenyl)amino]-7-[2-(2,2-dimethyl-6-oxo-morpholin-4-yl)-ethoxy]-6-[(S)—(tetrahydrofuran-2-yl)methoxy]-chinazoline
4-[(3-Chlor-4-fluor-phenyl)amino]-6-{2-[4-(2-oxo-morpholin-4-yl)-piperidin-1-yl]-ethoxy}-7-methoxy-chinazoline
4-[(3-Chlor-4-fluor-phenyl)amino]-6-[1-(tert.-butyloxycarbonyl)-piperidin-4-yloxy]-7-methoxy-chinazoline
4-[(3-Chlor-4-fluor-phenyl)amino]-6-(trans-4-amino-cyclohexan-1-yloxy)-7-methoxy-chinazoline
4-[(3-Chlor-4-fluor-phenyl)amino]-6-(trans-4-methansulfonylamino-cyclohexan-1-yloxy)-7-methoxy-chinazoline
4-[(3-Chlor-4-fluor-phenyl)amino]-6-(tetrahydropyran-3-yloxy)-7-methoxy-chinazoline
4-[(3-Chlor-4-fluor-phenyl)amino]-6-(1-methyl-piperidin-4-yloxy)-7-methoxy-chinazoline
4-[(3-Chlor-4-fluor-phenyl)amino]-6-{1-[(morpholin-4-yl)carbonyl]-piperidin-4-yl-oxy}7-methoxy-chinazoline
4-[(3-Chlor-4-fluor-phenyl)amino]-6-{1-[(methoxymethyl)carbonyl]-piperidin-4-yl-oxy}-7-methoxy-chinazoline
4-[(3-Chlor-4-fluor-phenyl)amino]-6-(piperidin-3-yloxy)-7-methoxy-chinazoline
4-[(3-Chlor-4-fluor-phenyl)amino]-6-[1-(2-acetylamino-ethyl)-piperidin-4-yloxy]-7-methoxy-chinazoline
4-[(3-Chlor-4-fluor-phenyl)amino]-6-(tetrahydropyran-4-yloxy)-7-ethoxy-chinazoline
4-[(3-Chlor-4-fluor-phenyl)amino]-6-((S)-tetrahydrofuran-3-yloxy)-7-hydroxy-chinazoline
4-[(3-Chlor-4-fluor-phenyl)amino]-6-(tetrahydropyran-4-yloxy)-7-(2-methoxy-ethoxy)-chinazoline
4-[(3-Chlor-4-fluor-phenyl)amino]-6-{trans-4-[(dimethylamino)sulfonylamino]-cyclohexan-1-yloxy}-7-methoxy-chinazoline
4-[(3-Chlor-4-fluor-phenyl)amino]-6-{trans-4-[(morpholin-4-yl)carbonylamino]-cyclohexan-1-yloxy}-7-methoxy-chinazoline
4-[(3-Chlor-4-fluor-phenyl)amino]-6-{trans-4-[(morpholin-4-yl)sulfonylamino]-cyclohexan-1-yloxy}-7-methoxy-chinazoline
4-[(3-Chlor-4-fluor-phenyl)amino]-6-(tetrahydropyran-4-yloxy)-7-(2-acetylamino-ethoxy)-chinazoline
4-[(3-Chlor-4-fluor-phenyl)amino]-6-(tetrahydropyran-4-yloxy)-7-(2-methansulfonylamino-ethoxy)-chinazoline
4-[(3-Chlor-4-fluor-phenyl)amino]-6-{1-[(piperidin-1-yl)carbonyl]-piperidin-4-yloxy}-7-methoxy-chinazoline
4-[(3-Chlor-4-fluor-phenyl)amino]-6-(1-aminocarbonylmethyl-piperidin-4-yloxy)-7-methoxy-chinazoline
4-[(3-Chlor-4-fluor-phenyl)amino]-6-(cis-4-{N-[(tetrahydropyran-4-yl)carbonyl]-N-methyl-amino}-cyclohexan-1-yloxy)-7-methoxy-chinazoline
4-[(3-Chlor-4-fluor-phenyl)amino]-6-(cis-4-{N-[(morpholin-4-yl)carbonyl]-N-methyl-amino}-cyclohexan-1-yloxy)-7-methoxy-chinazoline
4-[(3-Chlor-4-fluor-phenyl)amino]-6-(cis-4-{N-[(morpholin-4-yl)sulfonyl]-N-methyl-amino}-cyclohexan-1-yloxy)-7-methoxy-chinazoline
4-[(3-Chlor-4-fluor-phenyl)amino]-6-(trans-4-ethansulfonylamino-cyclohexan-1-yloxy)-7-methoxy-chinazoline
4-[(3-Chlor-4-fluor-phenyl)amino]-6-(1-methansulfonyl-piperidin-4-yloxy)-7-ethoxy-chinazoline
4-[(3-Chlor-4-fluor-phenyl)amino]-6-(1-methansulfonyl-piperidin-4-yloxy)-7-(2-methoxy-ethoxy)-chinazoline
4-[(3-Chlor-4-fluor-phenyl)amino]-6-[1-(2-methoxy-acetyl)-piperidin-4-yloxy]-7-(2-methoxy-ethoxy)-chinazoline
4-[(3-Chlor-4-fluor-phenyl)amino]-6-(cis-4-acetylamino-cyclohexan-1-yloxy)-7-methoxy-chinazoline
4-[(3-Ethinyl-phenyl)amino]-6-[1-(tert.-butyloxycarbonyl)-piperidin-4-yloxy]-7-methoxy-chinazoline
4-[(3-Ethinyl-phenyl)amino]-6-(tetrahydropyran-4-yloxy]-7-methoxy-chinazoline
4-[(3-Chlor-4-fluor-phenyl)amino]-6-(cis-4-{N-[(piperidin-1-yl)carbonyl]-N-methyl-amino}-cyclohexan-1-yloxy)-7-methoxy-chinazoline
4-[(3-Chlor-4-fluor-phenyl)amino]-6-(cis-4-{N-[(4-methyl-piperazin-1-yl)carbonyl]-N-methyl-amino}-cyclohexan-1-yloxy)-7-methoxy-chinazoline
4-[(3-Chlor-4-fluor-phenyl)amino]-6-{cis-4-[(morpholin-4-yl)carbonylamino]-cyclohexan-1-yloxy}-7-methoxy-chinazoline
4-[(3-Chlor-4-fluor-phenyl)amino]-6-{1-[2-(2-oxopyrrolidin-1-yl)ethyl]-piperidin-4-yloxy}-7-methoxy-chinazoline
4-[(3-Chlor-4-fluor-phenyl)amino]-6-{1-[(morpholin-4-yl)carbonyl]-piperidin-4-yloxy}-7-(2-methoxy-ethoxy)-chinazoline
4-[(3-Ethinyl-phenyl)amino]-6-(1-acetyl-piperidin-4-yloxy)-7-methoxy-chinazoline
4-[(3-Ethinyl-phenyl)amino]-6-(1-methyl-piperidin-4-yloxy)-7-methoxy-chinazoline
4-[(3-Ethinyl-phenyl)amino]-6-(1-methansulfonyl-piperidin-4-yloxy)-7-methoxy-chinazoline
4-[(3-Chlor-4-fluor-phenyl)amino]-6-(1-methyl-piperidin-4-yloxy)-7(2-methoxy-ethoxy)-chinazoline
4-[(3-Chlor-4-fluor-phenyl)amino]-6-(1-isopropyloxycarbonyl-piperidin-4-yloxy)-7-methoxy-chinazoline
4-[(3-Chlor-4-fluor-phenyl)amino]-6-(cis-4-methylamino-cyclohexan-1-yloxy)-7-methoxy-chinazoline
4-[(3-Chlor-4-fluor-phenyl)amino]-6-{cis-4-[N-(2-methoxy-acetyl)-N-methyl-amino]-cyclohexan-1-yloxy}-7-methoxy-chinazoline
4-[(3-Ethinyl-phenyl)amino]-6-(piperidin-4-yloxy)-7-methoxy-chinazoline
4-[(3-Ethinyl-phenyl)amino]-6-[1-(2-methoxy-acetyl)-piperidin-4-yloxy]-7-methoxy-chinazoline
4-[(3-Ethinyl-phenyl)amino]-6-{1-[(morpholin-4-yl)carbonyl]-piperidin-4-yloxy}-7-methoxy-chinazoline
4-[(3-Chlor-4-fluor-phenyl)amino]-6-{1-[(cis-2,6-dimethyl-morpholin-4-yl)carbonyl]-piperidin-4-yloxy}-7-methoxy-chinazoline
4-[(3-Chlor-4-fluor-phenyl)amino]-6-{1-[(2-methyl-morpholin-4-yl)carbonyl]-piperidin-4-yloxy}-7-methoxy-chinazoline
4-[(3-Chlor-4-fluor-phenyl)amino]-6-{1-[(S,S)—(2-oxa-5-aza-bicyclo[2.2.1]hept-5-yl)carbonyl]-piperidin-4-yloxy}-7-methoxy-chinazoline
4-[(3-Chlor-4-fluor-phenyl)amino]-6-{1 [(N-methyl-N-2-methoxyethyl-amino)carbonyl]-piperidin-4-yloxy}-7-methoxy-chinazoline
4-[(3-Chlor-4-fluor-phenyl)amino]-6-(1-ethyl-piperidin-4-yloxy)-7-methoxy-chinazoline
4-[(3-Chlor-4-fluor-phenyl)amino]-6-{1-[(2-methoxyethyl)carbonyl]-piperidin-4-yloxy}-7-methoxy-chinazoline
4-[(3-Chlor-4-fluor-phenyl)amino]-6-{1-[(3-methoxypropyl-amino)-carbonyl]-piperidin-4-yloxy}-7-methoxy-chinazoline
4-[(3-Chlor-4-fluor-phenyl)amino]-6-[cis-4-(N-methansulfonyl-N-methyl-amino)-cyclohexan-1-yloxy]-7-methoxy-chinazoline
4-[(3-Chlor-4-fluor-phenyl)amino]-6-[cis-4-(N-acetyl-N-methyl-amino)-cyclohexan-1-yloxy]-7-methoxy-chinazoline
4-[(3-Chlor-4-fluor-phenyl)amino]-6-(trans-4-methylamino-cyclohexan-1-yloxy)-7-methoxy-chinazoline
4-[(3-Chlor-4-fluor-phenyl)amino]-6-[trans-4-(N-methansulfonyl-N-methyl-amino)-cyclohexan-1-yloxy]-7-methoxy-chinazoline
4-[(3-Chlor-4-fluor-phenyl)amino]-6-(trans-4-dimethylamino-cyclohexan-1-yloxy)-7-methoxy-chinazoline
4-[(3-Chlor-4-fluor-phenyl)amino]-6-(trans-4-{N-[(morpholin-4-yl)carbonyl]-N-methyl-amino}-cyclohexan-1-yloxy)-7-methoxy-chinazoline
4-[(3-Chlor-4-fluor-phenyl)amino]-6-[2-(2,2-dimethyl-6-oxo-morpholin-4-yl)-ethoxy]-7-[(S)—(tetrahydrofuran-2-yl)methoxy]-chinazoline
4-[(3-Chlor-4-fluor-phenyl)amino]-6-(1-methansulfonyl-piperidin-4-yloxy)-7-methoxy-chinazoline
4-[(3-Chlor-4-fluor-phenyl)amino]-6-(1-cyano-piperidin-4-yloxy)-7-methoxy-chinazoline
   optionally, in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate.

Examples of preferred dopamin antagonists which may be mentioned include Bromocriptine, Cabergoline, Alpha-Dihydroergocryptine, Lisuride, Pergolide, Pramipexole, Roxindole, Ropinirole, Talipexole, Terguride and Viozane, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate.

Examples of preferred antiallergic agents which may be mentioned include Epinastine, Cetirizine, Azelastine, Fexofenadine, Levocabastine, Loratadine, Mizolastine, Ketotifene, Emedastine, Dimetindene, Clemastine, Bamipine, Cexchlorpheniramine, Pheniramine, Doxylamine, Chlorphenoxamine, Dimenhydrinate, Diphenhydramine, Promethazine, Ebastine, Desloratidine and Meclozine, optionally in racemic form, as enantiomers, diastereomeres or as pharmacologically acceptable salts, solvates or hydrates. Preferred are salts selected from the group consisting of hydrochloride, hydrobromide, hydroiodide, hydrosulfate, hydrophosphate, hydromethansulfonate, hydronitrate, hydromaleate, hydroacetate, hydrocitrate, hydrofumarate, hydrotartrate, hydrooxalate, hydrosuccinate, hydrobenzoate und hydro-p-toluenesulfonate.

Moreover, inhalable macromolecules can be used as pharmacologically active substances, as disclosed in European Patent Application EP 1 003 478 A1 or Canadian Patent Application CA 2297174 A1.

Moreover, the compound could be from the group of derivates of ergotalcaloids, triptane, CGRP-antagonists, phosphodiesterase-V-inhibitors, optionally in the form of the racemates, the enantiomers, the diastereomers and optionally the pharmacologically acceptable acid addition salts and the hydrates thereof.

As derivates of alkaloides: dihydroergotamine, ergotamine.

What is claimed is:

1. Method of atomizing a dry medicament formulation, wherein the medicament formulation is first dissolved by a liquid solvent and then the solvent is atomized with the dissolved medicament formulation.

2. Method according to claim 1, wherein the medicament formulation is dissolved in a continuous flow of the solvent.

3. Method according to claim 1, wherein the medicament formulation is dissolved in a discontinuous flow of the solvent.

4. Method according to claim 1, wherein a flow of the solvent is produced by at least one of gas pressure, pump pressure, capillary force, and hydrostatically.

5. An inhaler comprising a store containing at least one dry medicament formulation, said inhaler including means whereby said at least one medicament formulation can be dissolved by a liquid solvent and the solvent can be atomized with the dissolved medicament formulation for inhalation.

6. Inhaler according to claim 5, including means such that the medicament formulation can only be dissolved by the solvent during or immediately before the atomizing process.

7. Inhaler according to claim 5, including means for allowing the solvent to flow continuously through the store to dissolve the medicament formulation.

8. Inhaler according to claim 5, including means for allowing the solvent to flow discontinuously through the store to dissolve the medicament formulation.

9. Inhaler according to claim 5, including a reservoir for the solvent.

10. Inhaler according to claim 5, wherein the inhaler comprises a pressure generator for atomizing the solvent under high pressure through a common outlet.

11. Inhaler according to claim 10, wherein the store is arranged upstream or downstream of the pressure generator.

12. Inhaler according to claim 5, wherein the store comprises a plurality of storage regions each containing a dose of the medicament formulation.

13. Inhaler according to claim 12, wherein each storage region has its own separate outlet associated therewith.

14. Inhaler according to claim 5, including means for delivering simultaneously or successively at least two different medicament formulations during an atomizing operation.

15. Inhaler according to claim 14, wherein the store comprises various storage regions for the different medicament formulations.

16. Inhaler according to claim 15, including means for allowing the solvents for said medicaments to flow through the different storage regions in parallel in order to dissolve the different medicament formulations.

17. Inhaler according to claim 12, wherein the store has separate storage cavities to form the storage regions, the storage cavities being capable of being opened individually.

18. Inhaler according to claim 5, wherein the store is exchangeably inserted in the inhaler.

19. Inhaler according to claim 5, wherein the store contains the medicament formulation in dried-up form.

20. Inhaler according to claim 5, wherein the store contains the medicament formulation in powder form, pellet form, tablet form or in the form of a lyophilizate.

21. Inhaler according to claim 10, wherein said common outlet is a nozzle.

22. Inhaler according to claim 15, including means for allowing the solvents for said medicaments to flow through the different storage regions in series in order to dissolve the different medicament formulations.

23. Inhaler according to claim 5, including means for causing the atomization to take place purely mechanically.

24. Store having at least one dry medicament formulation for an inhaler, wherein the medicament formulation is dried up in the store.

25. Store according to claim 24, wherein the store comprises at least one storage region, said storage region including means for holding the medicament formulation in liquid or dissolved form before drying.

26. Store according to claim 25, wherein the storage region includes means for at least one of evening out the surface coverage and increasing the surface area.

27. Store according to claim 25, including means whereby each storage region can be filled or covered automatically by means of capillary forces with the medicament formulation in liquid form before drying up.

28. Store according to claim 24, comprising a plurality of storage regions each containing a dose of the medicament formulation.

29. Store according to claim 28, wherein each storage region has a separate outlet associated therewith.

30. Store according to claim 24, including separate storage cavities forming storage regions, said storage cavities being capable of being opened individually.

31. Store according to claim 24, wherein the dried-up medicament formulation forms one of a preferably uniform surface layer, a lyophilizate and a powder.

32. Store according to claim 24, wherein the dried-up medicament formulation is mechanically or chemically bound in the store.

33. Store according to claim 24, wherein the dried-up medicament formulation is substantially loose in the store.

34. Store according to claim 24, wherein the dried-up medicament formulation includes at least one active substance and contains carrier particles to which the active substance is bound.

35. Store according to claim 24, wherein the store is hermetically sealed to be at least substantially fluidtight and gastight after the drying of the medicament formulation.

36. Store according to claim 24, including means for allowing a solvent to flow through the store for dissolving the medicament formulation.

37. Store according to claim 36, including a reservoir for the solvent.

38. Store according to claim 24, wherein the store comprises attachment ports for the entry and exit of solvent.

39. Store according to claim 24, wherein the store contains multiple different dried-up medicament formulations.

40. Store according to claim 39, including different storage regions containing the different medicament formulations.

41. Store according to claim 40, including means for allowing a solvent to flow through the different storage regions in parallel or series in order to dissolve the various medicament formulations.

42. Store according to claim 24, wherein the store is constructed as a cartridge.

43. Store according to claim 24, wherein the store is constructed as a container.

44. Store according to claim 24, wherein the store is constructed as a blister.

45. Store according to claim 24, wherein the store is constructed as a foil.

46. Store according to claim 24, wherein the store is constructed as a microfluidic platform.

47. Store according to claim 24, wherein the store is constructed as a strip.

48. Store according to claim 24, wherein the store is constructed as a disc.

49. Store according to claim 24, wherein the dried-up medicament formulation includes at least one active substance and contains carrier particles with which the active substance is coated.

50. Store according to claim 36, including a plurality of reservoirs with a pre-metered solvent.

51. Store according to claim 26, wherein said means comprises a microstructured surface.

52. Store according to claim 26, wherein said means comprises a hydrophilic or hydrophobic section.

53. Store according to claim 26, wherein said means comprises edge structures.

54. Method of preparing at least one dry medicament formulation for an inhaler, comprising the steps of filling the medicament formulation in liquid or dissolved form into a store, and drying and sealing the medicament formulation in said store.

55. Method according to claim 54, wherein the filling of the store is effected by means of gas pressure.

56. Method according to claim 54, wherein the filling of the store is effected by means of pump pressure.

57. Method according to claim 54, wherein the filling of the store is effected by means of capillary force.

58. Method according to claim 54, wherein the filling of the store is effected hydrostatically.

59. Method according to claim 54, wherein the store is filled with one of a plurality of doses and with different medicament formulations.

60. Method according to claim 54, wherein the medicament formulation is dried by air drying.

61. Method according to claim 54, wherein the medicament formulation is dried by freeze drying.

62. Method according to claim 54, wherein the medicament formulation is dried by negative pressure.

63. Method according to claim 54, wherein the store is, after drying, hermetically sealed in fluidtight and gastight manner.

64. Method for producing an aerosol from a dried-up medicament formulation, comprising the step of expelling the dried-up medicament formulation by means of a gas current.

65. Method according to claim 64 wherein the dried-up medicament formulation is atomized by means of a gas current.

66. Method according to claim 64, wherein said dried-up medicament formulation is expelled by an air current produced by a user breathing in.
